(19) United States
(12) Patent Application Publication
(10) Pub. No.: US 2007/0259053 A1
(43) Pub. Date: Nov. 8, 2007

(54) Inhibition of nephrotoxicity induced by Trasylol® and like agents

(76) Inventor: William M. Selenke, Cincinnati, OH (US)

Correspondence Address: William M. Selenke, 5 Drummond St., Cincinnati, OH, 45218, US

(21) Appl. No.: 11/796,213

(22) Filed: Apr. 27, 2007

Related U.S. Application Data
(60) Provisional application No. 60/796,785, filed on May 2, 2006.

(51) Int. Cl. A61K 38/16; A61K 31/455; A61K 31/4415; A61K 31/4745; A61K 31/51; A61K 33/24

(52) U.S. Cl. 424/649; 514/350; 514/356; 514/276; 514/2; 514/305

(57) ABSTRACT

Increased positive charge of compounds or agents leads to increased glomerular filtration, increased binding by positively charged agents by the cells of the proximal tubules and decreased digestion of the reabsorbed materials. The present invention concerns the use of non-toxic positively charged materials to saturate binding sites on the proximal tubular brush border. If the binding sites on the brush border are saturated, then there should be decreased binding and absorption of the toxic agents by the proximal tubular renal cells. Specifically the invention is the use of combination of therapeutic positively charged agents comprising one or more of the following compounds; pyridoxal, niacin, thiamine and their relatively non-toxic analogs, amprolium, and isoniazid. These compounds are administered prior to the dosing of such positively charged therapeutics such as Trasylol® (aprotinin), cisplatin. or antimicrobiological peptides such as reported in patent application publication 2005/0065072. The predosing of one or more of the following compounds; pyridoxal, niacin, thiamine and their relatively non-toxic analogs, amprolium, and isoniazid inhibits the renal effects of Trasylol® (aprotinin), cisplatin or antimicrobiological peptides such as reported in patent application publication 2005/0065072. Likewise, a combination of therapeutic positively charged agents comprising one or more of the following compounds; pyridoxal, niacin, thiamine and their relatively non-toxic analogs, amprolium, and isoniazid, when administered with or without quinine for the treatment of malaria will inhibit the renal effects of malaria or such agents will inhibit renal failure caused by myoglobin released by crush injury

Priority claimed to provisional application No. 60/796,785

### FIELD OF INVENTION

This invention is concerned with prevention of nephrotoxicity of positively charged pharmacological agent such as Trasylol®, quinine, and antibiotics such as antimicrobiological peptides such as reported in patent application publication 2005/0065072. More generally it is concerned with the inhibition of nephrotoxicity which nephrotoxicity is caused by positively charged agents. The inhibitory agents are non-toxic positively charged agents.

### BACKGROUND OF INVENTION

Modern medicine would be difficult without effective pharmaceuticals. However, it is not generally appreciated that not only the ordinary medical use of, but also the discovery of pharmaceuticals is both an art and a science. The discovery of new medicinal agents depends on both luck and a proper theoretical understanding of the causes of disease. The classic example is the effective treatment of bacterial infectious diseases. Once the germ theory of disease postulated by Pasteur was accepted and once it was understood that certain agents such as penicillin could kill bacteria the art of treating infectious diseases became quite effective because it was relatively easy, although costly, to find agents that killed or inhibited bacteria. Yet most drugs have side effects such as kidney failure.

Chemically caused nephrotoxicity is of fundamental importance to the practicing physician as well as the research toxicologist. Even though the kidney is affected by general toxins which affect other organs of the body, commonly the first signs of toxicity of a chemical are manifest in the kidney. This is for several reasons. The kidney receives about 5% of the total blood flow, yet the kidney constitutes 1 percent of the body weight. More importantly, the kidney is involved in the process of resorption of useful small chemicals such as amino acids and glucose as well as the transport of various cellular metabolites as well as the elimination of various ingested toxic chemicals. For these reasons many chemicals which do not cause acute or chronic toxicity in other parts of the body will cause either acute renal failure or chronic nephritis.

An hypotheses for the prevention of nephrotoxicity was published as U.S. Pat. No. 4,654,325 (1). This patent outlines the means of preventing kidney toxicity especially damage caused by aminoglycosides. The recent national publicity of the nephrotoxicity of Trasylol® (aprotinin) suggested to this inventor that he should revisit this subject and revise his hypothesis.

The revised hypotheses states that by administrating a positively charged agent at effective pharmacological doses of such non toxic agent will saturate the negatively charged binding sites on the luminal membranes of the proximal tubular cells. Such agents by saturating the proximal luminal binding sites will prevent the binding and thus reabsorption of toxic positively charged chemicals. Such toxic chemicals will pass into the urine and will not be absorbed after filtration by the glomerulus of the kidney.

The normal pI or charge of proximal tubular or luminal membranes (certainly the charges of isolated renal brush border membranes) is about 4.5. Thus any chemical which has an isoelectric point much above 4.5 is considered by the kidney is positively charged. As a practical matter, there are few pharmacological agents that cause renal toxicity that have an isoelectric point below about the neutral 7.5. However, Bence Jones proteins of multiple myeloma have an increased pI compared to the normal protein pI of about 4.5. Bence Jones proteins cause renal failure over time. Secondly, myoglobin in the blood circulation after crush injury can cause renal failure. Finally, hemoglobin which is released when the red blood cells are lysed, such as by certain types of malaria, can cause renal failure. This coupled with the fact that the traditional pharmaceutical treatment for malaria, quinine, has a positive charge and of can (of itself) cause renal damage. Thus in malaria there can be potentially a double problem. Quinine, which is the must useful agent to treat malaria, can cause renal damage. Hemoglobin from the lysed cells likewise causes renal damage. Thus the agents of the present invention are likely to be useful to prevent toxicity induced by the positively charged quinine as well as the nephrotoxicity caused by the release of hemoglobin especially in “Black Water” fever or falciparum malaria.

To add understanding of this process in my doctoral dissertation (2), I postulated the mechanism of action of calcium in biochemical reactions. I will quote from the dissertation:

“As to be recalled, Just and Haberman (3) were able to abolish the binding of Trasylol (aprotinin) by changing its pI from 10.5 to 7.0. Similarly, the decreased amounts of pI 10.5 Trasylol retained by BBM by removing the sialic acid residues on the BBM. This removal of sialic acid residues increased the pI of the BBM from about 4.5 to 7.0.

For convenience, let (+) represent net positive charge on a structure, and let (−) represent a net negative charge. Thus, if we only consider the effects of charge we can define the reaction of Trasylol to BBM as Trasylol (+)(−) BBM. Since lysozyme has a net positive charge at neutral pH, the reaction of lysozyme with BBM can similarly be represented as lysozyme (+)(−) BBM.

It is to be recalled that CdMT (Cadmium metallothionein) has a pI of about 4.0. Thus, it is negatively charged at neutral pH e.g., CdMT (−). The reaction of CdMT with BBM at neutral pH is represented as CdMT (−)(−) BBM. Two negative charges repulse each other so that only minimal amounts of CdMT react with BBM without added divalent cations. A divalent cation such as Ca⁺⁺ could provide a bridge charge to bind CdMT to BBM, e.g., CdMT(−)(+) Ca(+)(−) BBM. Further, calcium decreased the binding of lysozyme to BBM. This decrease in binding can be represented as follows: lysozyme (+)(+) Ca(+)(−) BBM. Thus, calcium could induce a net positive charge which would repulse the positively charged lysozyme. Similarly, at pH 6.5 calcium decreased the amounts of pI 8.5 albumins retained by BBM compared to samples that did not have added divalent cations. This reaction would, of course, be represented as albumin (+)(+) Ca(+)(−) BBM.” End of dissertation citation.

The above information was discovered by investigating the binding reactions of positively charged agents with renal proximal tubular luminal membranes. However, it would be useful to review briefly the role of positive charge on the filtration at the glomerular membranes as well as consider the metabolism of positively charged agents once they are absorbed by the proximal tubules.

Earlier basic research on the mechanisms of kidney filtration used non-physiological chemicals or proteins of specific size and usually increased charge such as dextrans or the pharmaceutical excipient, povidone (polyvinylpyrrolidone), or various proteins such as egg white lysozyme or horseradish peroxidase which are not usually presented to the kidney in normal physiological situations. See the reviews of Brenner et al, (4) and Rennke and Venkatachalam (5). In summary, the increase in charge led to an increased glomerular filtration. By increasing the charge of the protein or other large molecules while keeping the same basic size caused a significant increase in the filtration of chemical entities. It is interesting to note that Bulger et al. (6) reported that there was a number of large pores in freeze dried fractured kidney of rats that received gentamicin, even though there was a decrease in glomerular filtration. In any case, the administration of proteins with increased isoelectric points led to an increase in the amounts of proteins, dextran, and povidone filtered by the kidney. It is not unreasonable to state that, in part, the toxicity of positively charged agents is caused by an increased filtration fraction of such agents.

The evidence is that once positively charged agents are filtered by the kidney, they bind avidly to the renal brush border membranes which is confirmed by autoradiography of light microscopy (7, 8) and by chemical analysis (9).

The increase in filtration fraction induced by positively charged proteins and the increased binding of the filtered agents to the luminal membranes is important in that these processes should lead to an increased load of positively charged agents in the proximal tubule. There are interesting observations (4,10) about the fate of such positively charged agents once they are in the proximal cell. Once positively charged agents are in the cell they remain a long time. In the case of aprotinin (Trasylol) 72 hours after administration its level peaked in the kidney (3) as noted by autoradiography. The same can be said for other positively charged proteins. In addition, reabsorbed gentamicin and other aminoglycosides remain in the proximal cell for extended periods (10).

In the mid-Seventies Dice and Goldberg (11) reported that proteins with increased isoelectric points are metabolized at a decreased rate which correlated with the isoelectric point. That is, proteins with increased isoelectric points resisted digestion to a much greater extent than proteins with normal pI. Thus, it is likely that by increasing the charge of proteins and other agents, the proteolytic enzymes and perhaps other enzymes are compromised leading to toxic reaction and cell death of the metabolically active proximal tubular cells.

This suggestion is supported by the work of Clyne et al. (12). They reported that renal damage of both human and in the rat correlated with the isoelectric point of the Bence Jones proteins. However, work by Sanders (13) suggested that this is oversimplified. However, other work with the proteolytic enzymes shows that rates of protein digestion is a function not only of isoelectric point, but also size and amino acid composition of such proteins (14). The resolution of this point will require the experimental comparison of protein digestion by various renal proteolytic enzymes to determine if the toxic effects of the various Bence Jones and myoglobin proteins are related with the speed with which they are digested by the kidney. In any case, if the proteins can not be absorbed by the proximal tubular cells, that may lessen kidney toxicity.

### The Binding of Positively Charged Agents by the Renal Brush Border

While the effects of positive charge on the renal filtration of positively charged toxins and the final disposition of them by the proximal tubular cells is of importance for renal toxicity, the presumption of this patent is that positive charge provided by agents other than the toxin in question will prevent the binding and absorption of the toxic agent. The second presumption is that positively charged agents non-toxic at pharmacological doses whether as a single agent or a cocktail of several of such non-toxic agents will prevent the absorption of such toxic agents by the proximal tubule and therefore greatly lessen the renal damage of positively charged agents. The first of these presumptions is amply shown by the literature. The second has some limited support. It is the hope that a systematic look at agents such as amprolium (15, 16), thiamine, nicotinic acid, pyridoxal 5′-phosphate (vitamin B6) (17, 18) niacin, isoniazid each alone or in various combinations will prevent much of the renal toxicity of various positively charged renal toxins such as the aminoglycosides, therapeutic heavy metals (gold and platinum) and various proteins such as the Bence Jones, myoglobin, and hemoglobin proteins.

### Summary of the Literature that Supports this Hypothesis

Keniston, et al. (17) reported that pyridoxal 5′-phsophate inhibited the toxic effects of the positively charged renal toxin, spermine (17) and gentamicin. This work with gentamicin was confirmed by Kacew (18).

Jerauld and Silverblatt (19) reported that a classic inhibitor of the organic base transport system, N′-methylnicotinamide, which is relatively non-toxic and transported inhibited the renal accumulation of gentamicin and increased the urinary excretion of gentamicin in rats.

Kirschbaum (20) and Humes et al. (21) reported that calcium is a competitive inhibitor of binding of gentamicin to isolated renal brush border membranes. The relationship between the calcium stimulated binding with isolated BBM and the whole animal effects as measured by animal survival and renal function held up as shown by Humes (21), and Bennett et al. (22)

It is to be noted that Bennett et al. (23) reported that sodium depleted rats had increased gentamicin toxicity and that Viau et al. (24) reported that cadmium treated animals (cadmium is a heavy metal nephrotoxin) reduced gentamicin accumulation in the renal cortex. Selenke (2) recorded that brush border membranes from chronic cadmium treated rabbits had an increased binding of cadmium metallothionein as did rabbit BBM in which cadmium was added to brush border membrane isolated from untreated rabbits suggesting that cadmium provided a positive charge on the renal brush border membranes.

Of interest is the effects of quinine. Quinine is a less significant renal toxin, although it is an ototoxin. In a limited series of experiments Whelton et al. (25) reported that quinine inhibited the renal absorption of gentamicin. Positively charged polylycine inhibited both the binding of gentamicin to isolated BBM and lessened the acute toxic effects of whole animal nephrotoxicity (26). Likewise, Josepovitz et al. (27) reported that tetralysine inhibited the uptake of gentamicin in rat renal cortex. Malis, et al. (28) reported that lysine had an additive effect with the renal toxic effects of gentamicin. Lysine itself is a nephrotoxin (29). Spermine is of itself a nephrotoxin (30). However, it inhibited the uptake of gentamicin by renal tubular BBM (31), as well as in vivo experiments. Further indication that there is a common factor in the various positively charged BBM binding is that the positively charged protein, aprotinin, inhibited the binding of gentamicin to isolated BBM (34), but of itself, aprotinin did not unambiguously inhibit the renal absorption of gentamicin in vivo (32).

Clonidine, an adrenergic antihypertensive agent which is positively charged and secreted by the kidneys (33,34), inhibited gentamicin induced renal toxic effects in the rat kidney (34). Clonidine protected against mercury induced acute renal failure (35, 36). It is to be noted that cisplatin and aminoglycosides have increased renal toxicity when given together (37,38).

The classic cationic transport inhibitors, quinine, cynamine, tetraethylamonium (TEA), and N′-methylnicotinamide all had effects on the renal handling of cisplatinin. As mentioned above, for purposes of this review, those agents since they are readily filtered and secreted in the proximal tubule lumen are considered to provide a net positive charge at that lumen brush border binding sites. Quinine in both the bird (39) and rat provided protection against the renal toxic effects of cisplatin. Williams and Hottendorf (40) reported that cisplatin inhibited the transport of N′-methylnicotinamide and tetraethylamonium (TEA) into vesicles of BBM. Nelson et al. (41) reported that cisplatin inhibited the uptake of TEA and p-aminohipputate by different mechanisms in the mouse kidney.

### Nephrotoxic Effects of Certain Proteins

Selenke (2) reported that Ca⁺⁺ inhibited the binding of both lysocyme and albumins of increased isoelectric points to the binding of BBM. In a series of microinfusion experiments Cojocel et al. (42) reported that cytochrome C ribonuclease, spermine, L-arginine, and L-lysine inhibited the renal accumulation of lysozyme. Myoglobin, which is a more neutral compound, was without effect in their system.

A modern cliché is the concept of Thomas Kuhn's “paradigm”. That is, a person considers a subject the way he is taught to look at it. A nephrologist thinks “organic base transport blocker” when he reads “quinine”, N′-methynicotinamide”, “tetraethylamonium”, and so forth. Thus, if these agents produce an effect on the kidney, the presumption is that the effect is caused by “transport blocking”. These chemicals are useful tools. If one is investigating the metabolic elimination of a new chemical, it is necessary to use them to determine if the chemical in question is eliminated in part by the organic transport system. The reality, of course, is that these agents are themselves transported into the lumen of the proximal cell. It is the presumption of this patent document that this excess positive charge will react with the negative charges on the luminal membranes and prevent the binding of positively charged nephrotoxins.

It is to be noted that not all of the results reported need to be forced onto the present hypothesis. For example, the work by Bennett et al. (23) with sodium depleted animals may be considered to be the effects of gentamicin on abnormal animals. The binding work of Selenke (2) had high levels of sodium chloride in the reaction media. Yet the binding effects of divalent cations were quite apparent. Second, it is interesting to report the effects of clonidine (32,33,34,35,36), an agent which is positively charged and transported by the kidney. These clonidine reports were found because they fit the hypothesis. However, since clonidine is an adrenergic agent, and therefore profoundly alters blood vessels, the nephroprotective effects may be independent of its charge.

What is missing is the systematic efforts to determine if the non-toxic vitamins pyridoxal, niacin, thiamine and their relatively non-toxic analogs, amprolium and isoniazid alone and in combination (and other such non-toxic positively charged agents filtered and secreted by the kidney) will prevent kidney damage caused by agents with a relative positive charge.

### SUMMERY OF THE INVENTION

It is known in the scientific medical literature that increased positive charge leads to increased glomerular filtration, increased binding by the cells of the proximal tubules and decreased digestion of the reabsorbed materials. The present invention concerns the use of non-toxic positively charged materials to saturate binding sites on the proximal tubular brush border. If the binding sites on the brush border are saturated, then there should be decreased binding and absorption of the toxic agents by the proximal tubular renal cells.

Specifically the invention is the use of combination of therapeutic positively charged agents comprising one or more of the following compounds; pyridoxal, niacin, thiamine and their relatively non-toxic analogs, amprolium, and isoniazid. These compounds are administered prior to the dosing of such positively charged therapeutics such as Trasylol® (aprotinin), cisplatin. or antimicrobiological peptides such as reported in patent application publication 2005/0065072. The predosing of one or more of the following compounds; pyridoxal, niacin, thiamine and their relatively non-toxic analogs, amprolium, and isoniazid inhibits the renal effects of Trasylol® (aprotinin), cisplatin or antimicrobiological peptides such as reported in patent application publication 2005/0065072. Likewise, a combination of therapeutic positively charged agents comprising one or more of the following compounds; pyridoxal, niacin, thiamine and their relatively non-toxic analogs, amprolium, and isoniazid, when administered with or without quinine for the treatment of malaria will inhibit the renal effects of malaria or myoglobin released by crush injury

I claim:

1) A combination of therapeutic positively charged agents comprising one or more of the following compounds; pyridoxal, niacin, thiamine and their relatively non-toxic analogs, amprolium, and isoniazid;
wherein said compounds are administered prior to the dosing of such positively charged therapeutics such as Trasylol® (aprotinin), cisplatin. or antimicrobiological peptides such as reported in patent application publication 2005/0065072;
which predosing inhibits the renal effects of Trasylol® (aprotinin), cisplatin or antimicrobiological peptides such as reported in patent application publication 2005/0065072.

2) A combination of therapeutic positively charged agents comprising one or more of the following compounds; pyridoxal, niacin, thiamine and their relatively non-toxic analogs, amprolium, and isoniazid;
wherein said compounds are administered with or without quinine for the treatment of malaria to inhibit the renal effects of malaria.

3) A combination of therapeutic positively charged agents comprising one or more of the following compounds; pyridoxal, niacin, thiamine and their relatively non-toxic analogs, amprolium, and isoniazid;
wherein said compounds are administered inhibit the renal effects myoglobin released by crush injury.
